(19) United States
(12) Patent Application Publication
(10) Pub. No.: US 2013/0171722 A1
(43) Pub. Date: Jul. 4, 2013

(54) METHOD AND APPARATUS FOR DELIVERY OF MOLECULES TO CELLS

(71) Applicants: Xianfeng CHEN, Tai Wei (HK); Wenjun ZHANG, Hong Kong (HK)

(72) Inventors: Xianfeng CHEN, Tai Wei (HK); Wenjun ZHANG, Hong Kong (HK)

(73) Assignee: CITY UNIVERSITY OF HONG KONG, Kowloon (HK)

(21) Appl. No.: 13/342,529

(22) Filed: Jan. 3, 2012

(51) Int. Cl. C12M 1/00

(52) U.S. Cl. 435/283.1

(57) ABSTRACT

The present invention is concerned with an apparatus for injection of a substance into a subject. The apparatus has an assembly including a plurality of elongate non-hollow micro-nanoneedles for delivering the substance into the subject, at least some of the micro-nanoneedles have a non-uniform diameter with a wider upper end and a narrower lower end and a length from 5-100 um, wherein the upper end has a diameter from 1-20 um and the lower end has a diameter from 50-400 nm, wherein the micro-nanoneedles are spaced apart with appropriate spacing therebetween such that when the micro-nanoneedles are penetrating cells in a penetration exercise each of the micro-nanoneedles may penetrate one cell only, and wherein the micro-nanoneedles are made from a relatively rigid material allowing the micro-nanoneedles to maintain rigidity during penetration and are configured to allow coating of the substance on the surface of the lower end.

### FIELD OF THE INVENTION

The present invention is concerned with a method and an apparatus for delivery of molecules, e.g. drug, gene, to cells.

### BACKGROUND OF THE INVENTION

Cell therapy has become very attractive in medical technology in recent years because it provides a unique opportunity to treat some certain diseases (e.g. human liver cirrhosis). In cell therapies, molecules, drugs or genes such as plasmid DNA, siRNA, miRNA, shRNA, nanoparticle or nanowire are to be delivered to the cell cytoplasm or nuclei in order that they become functional and therapeutic. Such molecules play important roles in medical treatment. However, these molecules are typically rather complicated and cannot penetrate cell membranes by simple diffusion. Various approaches have been suggested to deliver these molecules to cells such as cell penetrating peptides, electroporation, ballistic nanoparticle delivery, viral vectors and nanoneedles. However, they suffer from different limitations.

The present invention seeks to address some of the problems in delivering molecules into a subject and in particular within cells, or at least to provide an alternative to the public.

### SUMMARY OF THE INVENTION

According to a first aspect of the present invention, there is provided an apparatus for injection of a substance into a subject, comprising an assembly including a plurality of elongate non-hollow micro-nanoneedles for delivering the substance into the subject, at least some of said micro-nanoneedles have a non-uniform diameter with a wider upper base end and a lower tip end and a length from 5-100 um, wherein the upper end has a diameter from 1-20 um and the lower end has a diameter from 50-400 nm, wherein the micro-nanoneedles are spaced apart with appropriate spacing therebetween such that when the micro-nanoneedles are penetrating cells in a penetration exercise each of them may penetrate one cell only, or at least to minimize multiple micro-nanoneedles penetrating one cells, and wherein the micro-nanoneedles are made from a relatively rigid material allowing the micro-nanoneedles to maintain rigidity during penetration and are configured to allow coating of the substance on surface of the lower end. The plurality of micro-nanoneedles may take the form of a micro-nanoneedle patch.

Preferably, the substance may be a bio-agent or may be selected from a group including drug molecules, gene molecules such as plasmid DNA, siRNA, miRNA, shRNA, nanoparticles and nanowires.

In an embodiment, the subject may be a sample of cells to be treated and collected on a substrate. The substrate may be a multi-well array of container.

In one embodiment, the micro-nanoneedles may be configured to adopt a generally conical profile. Conically shaped micro-nanoneedles can facilitate penetration of the low end into cells. In a specific embodiment, the lower end of the micro-nanoneedles may be pointy and/or sharp.

In a specific embodiment, lower end may have a diameter from 50-100 nm. Depending on the cell type on which the system is to be used, each of the micro-nanoneedles may be spaced apart from an adjacent micro-nanoneedle by a distance from 5-50 um.

The micro-nanoneedle patch together can treat a larger number of cells in one go. In a specific embodiment, the micro-nanoneedle patch may have a micro-nanoneedle density of 10⁵-10⁶ micro-nanoneedles per cm².

The apparatus may comprise means for applying the micro-nanoneedle patch to the subject at a speed of 0-5 m/s.

In an embodiment, one or all of the micro-nanoneedles may be formed from growing through deposition of diamond and cBN films followed by a post-deposition reactive ion etching treatment.

According to a second aspect of the present invention, there is provided a method of manufacture of an apparatus as described above.

According to a third aspect of the present invention, there is provided a method of manufacture of an apparatus for injection of a substance into a subject, the apparatus comprising one or more micro-nanoneedles for use in delivering the substance into the subject, the method comprising steps of growing the micro-nanoneedles through deposition of diamond and cBN films, performing post-deposition reactive ion etching treatment and coating the substance on a lower end of the nanoneelde(s) for delivery thereof into the subject.

In an embodiment, the films may be pre-coated through a fold etching mask. The films may be further etched in hydrogen/argon-based microwave plasma with the assistance of a negative substrate bias.

The substance may be coated on the lower end by way of dry coating. Alternatively, the substance may be coated on the lower end by way of disulphide bonding.

According to a fourth aspect of the present invention, there is provided an apparatus for injection of a substance into a subject made according to the method of the third aspect of the invention.

### BRIEF DESCRIPTION OF DRAWING(S)

Embodiments of the present invention are now described, by way of example only, with reference to the following drawing in which:—

FIG. 1 is a schematic diagram showing delivery of a substance to within living cells by an apparatus according to an embodiment of the present invention;

FIG. 2 is a schematic diagram of a micro-nanoneedle patch for use in the apparatus of FIG. 1 according to an embodiment of the present invention; and

FIG. 3 is a schematic diagram showing attachment of a substance to a surface of a micro-nanoneedle, e.g. to surface of the micro-nanoneedles as shown in FIG. 2, according to an embodiment of the present invention.

### DETAILED DESCRIPTION OF EMBODIMENTS OF THE INVENTION

In the preamble of the specification, it has been discussed that various approaches have been proposed to deliver biomolecules or therapeutic molecules to cells for treatment purpose. For example, the use of cell penetrating peptides (CPPs), electroporation, viral vectors and nanoneedles has been suggested before.

The use of CPP is one of the approaches to deliver different molecules into cells because this approach can be carried out in a non-invasive way. CPPs are short peptides that facilitate cellular uptake of various molecules. The molecules to be delivered are associated with the peptides either through covalent bonds or non-covalent interactions. While this method has great potential in that the CPPs act as delivery vehicles, there are limitations associated therewith. For example, studies have shown that the method is not functional when applied in certain some cell lines (e.g. Madine Darby canine kidney cells). Further, studies have shown that the method has limited in-vivo applicability.

Electroporation can be used to increase the permeability of cell plasma membrane by applying an external electrical field so that a dug can enter cells. However, this approach often causes cell death.

Viral vectors can be used to deliver genes into cells. During application, the genes in the virus that cause disease is removed and replaced with genes encoding the desired effect. However, there are serious limitations such as infecting healthy cells, causing harmful mutations to DNA or even cancer. Studies have shown that they can even lead to death of a patient in a clinical trial.

Individual nanoneedles may be used separately. It has been found that nanoneedles with diameter less than 400 nm can pierce cell membranes without causing irreversible destruction of the membrane with 1 hour insertion. Nanoneedles have been demonstrated to deliver fluorescent quantum dots into the cytoplasm and nucleus of living cells, but its shortcoming is that it is very time consuming if one needs to treat a large number of cells.

To increase the efficacy, arrays of bundles of nanotubes have been also employed. One nanotube however would not be strong enough, although a bundle of 5-10 nanotubes together can be sufficiently strong. The shortcomings of this technique include: (1) the tip diameter of each tube is usually one micrometer or above, (2) the bundled nanoneedles will have relatively uniform diameter for their whole length and it is very difficult to maintain strength for piercing large cell membranes to deliver drugs to cell nuclei when the required nanoneedle length is long (e.g., close to 100 um), (3) since the nanotube diameter is very small (below 200 nm or even 100 nm or even 70 nm), it is difficult to absorb a reasonable high amount of large drug molecules into the tube (for example, plasmid DNA length from 0.3 to 66 nm, chromosome DNA is much larger).

To address these limitations, the present invention has been developed to provide a novel technique using arrays of micro-nanoneedles to deliver a substance to a subject. In a specific embodiment, the present invention is concerned with an apparatus having an assembly with a plurality of the micro-nanoneedles for delivering the substance into the subject, each of the micro-nanoneedles has non-uniform diameter with a wider upper end and a narrower lower end and a length from 5-100 um. This length ensures that the needles are sufficiently long to reach within the interior of the cells. The upper end has a diameter from 1-20 um and the lower end has a diameter from 50-400 nm. Studies have shown that while the tip diameter may range from 50-400 nm (which is a workable range for most cell types) in that the needles with this tip dimension do not impair the cell viability or membrane integrity, the tip diameter may range from 50-100 nm. It is to be noted that the micro-nanoneedles are spaced apart such that different micro-nanoneedles in the assembly are for penetrating different cells in a cell sample in a penetration exercise, or at least to minimize the number of micro-nanoneedles penetrating one cell. This is advantageous in that it can significantly speed up the delivery of the substance into the subject.

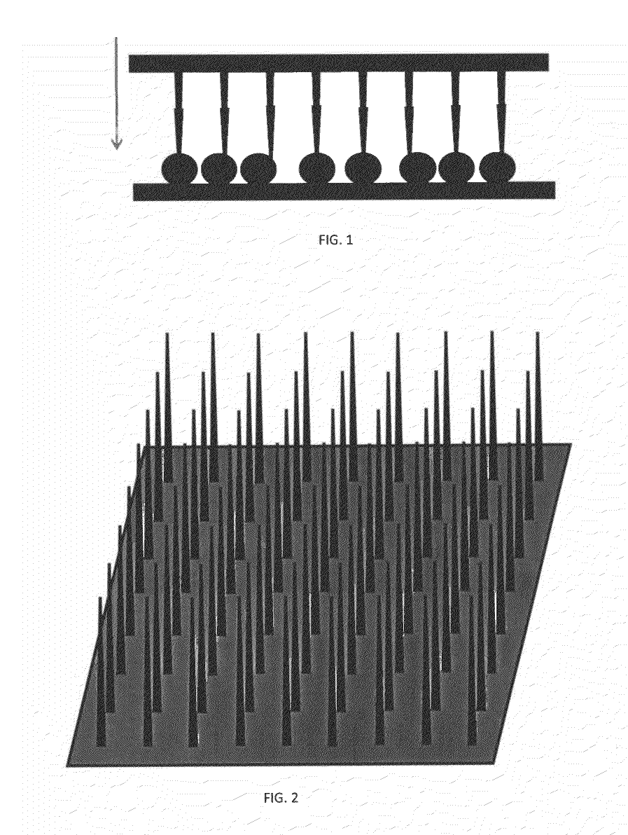

The micro-nanoneedles are configured such that they are non-hollow. In other words, a substance to be delivered by the needles does not pass through the needles. The non-hollow configuration means that when compared with tubes or pipes the rigidity of the needles is much improved. Even when the needles are constructed to be relatively thin yet sufficient rigidity thereof is still preserved. The micro-nanoneedles are rigid enough such that they do not rely on bundling nanoneedles together in a penetration exercise. In use, the cells are collected on a substrate which may be a multi-well array of a container. Alternatively, the substrate may be a planar member for providing a surface on which the cells rest during the penetration. FIG. 1 shows the resting of the cells on the substrate surface.

The micro-nanoneedles may be made from a material that allows them to maintain rigidity during a subject penetration process. The material which may be used will be discussed further below.

FIG. 1 demonstrates that the micro-nanoneedles are configured to adopt a generally conical profile. The lower end of the micro-nanoneedles is pointy and sharp. This profile facilitates the piercing of cell membrane by the tip of the micro-nanoneedles. In the embodiment shown in FIG. 1, the diameter of the tip of the needles is about 100 nm. However, as discussed above as long as the tip diameter is from 50-400 nm or preferably 50-100 nm, the needles can still satisfactorily pierce through the cell membrane and deliver the substance without causing irreversible damage to the cell membrane. In this embodiment, the micro-nanoneedles are spaced from each other and the spacing between adjacent micro-nanoneedles is from 5-50 um depending on the types of cells. It can be seen from FIG. 1 that the plurality of micro-nanoneedles together takes the form of a micro-nanoneedle patch. The micro-nanoneedle patch has a micro-nanoneedle density of 10⁵-10⁶ micro-nanoneedles per cm². With this spacing, the micro-nanoneedles are densely packed enough to allow piercing of large number of cells for many cell types in one go and yet one particular cell would not be pierced by more than one micro-nanoneedle. This can facilitate the speed of treating or delivering of a substance into the cells in an efficient manner.

The apparatus is provided with means such that the micro-nanoneedle patch can be applied to the cells to be treated at a speed of 0-5 m/s. With this feature, treatment of a large number of cells can be performed efficiently in an automated manner. Such means, e.g. automated actuation means, are well known in the art of bio-instrumentation.

FIG. 2 is a schematic diagram of a micro-nanoneedle patch. The micro-nanoneedles can be made of diamond. In one example, the diamond micro-nanoneedle structures was grown from deposition of diamond and cBN films followed by a post-deposition reactive ion etching (RIE) treatment. The films were pre-coated through a gold etching mask and then etched in hydrogen/argon-based microwave plasma with the assistance of a negative substrate bias. Through this process, micro-nanoneedle structures can be fabricated. The geometry, density and aspect ratio of these micro-nanoneedle structures can be adjusted by controlling the etching mask design, microwave power, substrate temperature, and etching duration.

After fabricating the micro-nanoneedle structures, the lower end, or tip, of the cones are to be treated such that they will carry a substance to be delivered into the cells. The substance may be attached to the tip in a number of different ways. In one embodiment, the substance, e.g. drugs or genes, may be coated onto the surface of the tip. In this particular embodiment, the substance is plasmid EGFP DNA and CdSe quantum dots (QDs). The plasmid EGFP DNA and CdSe QDs are dry-coated on the surface of micro-nanoneedles for subsequent delivery. One approach of coating the DNA or QDs onto the micro-nanoneedles is by use of a viscosity enhancer (e.g., methylcellulose, honey). Specifically, a coating solution is prepared. For example, the solution may contain either pEGFP DNA or CdSe QDs, methylcellulose as viscosity enhancer, and poloxamer (F-68) as surfactant. Then the DNA or QDs can be coated onto micro-nanoneedles using dip-coating or gas jet drying coating methods (e.g., WO2009/079712, US2011/0059150, WO2010/042996). With these methods, the coating on the needles is sufficiently robust during insertion and yet it can be quickly released (dissolved) within hydrated environment after insertion into the interior of cells.

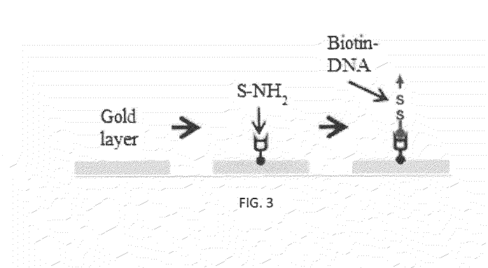

Another approach of coating the DNA or QDs is by way of using disulfide bonding. With this approach, a thin layer of gold (˜10 nm) is firstly deposited on the micro-nanoneedle structure. Then, a NH₂-terminated self-assembly monolayer is formed by the chemisorption of thiols on gold. The biotinylated DNA containing a disulfide bond can then conjugate to the surface. These procedures are illustrated in FIG. 3. As far as QD coating is concerned, a third step will be conjugating sulfo-NHS—SS-biotin and introducing a further step to bind streptavidin-coated CdSe QDs by the specific binding of streptavidin and biotin. Streptavidin-coated CdSe QDs is a product that can be purchased on the market. Once the micro-nanoneedle is in water or inserted into an interior cellular environment, DNA will be released as most disulfide bonds are reduced into thiol groups (R—SS—R+2H++2e-□R—SH+SH—R).

It is to be noted that the above embodiment of a delivery apparatus does not require the use of a reservoir for supplying the substance to be delivered. The free of such reservoir allows a user to deliver of the substance to target cells more easily and efficiently because the user does not need to fill up a liquid substance to the reservoir. It is also to be noted that the needles are solid and this removes complications of engineering a very small channel within tube or pipe structures and delivering substances via such structures.

In an alternative embodiment, there is provided a system in which the micro-nanoneedles are free of coating of a substance to be delivered into a subject. In such embodiment, the apparatus comprises a micro-nanoneedle patch for introducing holes to the membrane of the cells of the subject suspended or immersed in a substance-containing fluid. During penetration of the cell membrane, the substance-containing fluid can enter and diffuse into the cells via the holes.

Yet in a different embodiment, there is provided a system in which the micro-nanoneedles when manufactured are not coated with a substance to be delivered into a subject. The substance is provided to the tip of the micro-nanoneedles just before they are to penetrate the cells. The may be achieved by first dipping a micro-nanoneedle patch into a solution containing the substance to be delivered to the subject (e.g. a drug). The dipping of the patch allows the drug to absorb to the surface of the tips and/or in between the tips. Then, the patch can be used to pierce the cells to deliver the drug to the interior of the cells.

It should be understood that the above only describes the preferred embodiments according to the present invention, and that modifications and alterations may be made thereto without departing from the spirit of the invention. Further, a skilled person in the art will be familiar with the following prior art which is incorporated into the description herein in their entirety by way of reference.
Foerg C, Merkle H P, J Pharm Sci (2008) 97:144-162.
Rubinsky B, Technol Cancer Res Treat (2007) 6:255-259.
Savulescu J, J Med Ethics (2001) 27:148-150.
Han S W, Nakamura C, et al. Biochem Bioph Res Co (2005) 332:633-639.
Yum K, Na S, et al. Nano Lett (2009) 9:2193-2198.
Zhang W J, Meng X M, et al. Appl Phys Lett (2003) 82
U.S. Pat. No. 6,261,554
EP1195440
U.S. Pat. No. 6,620,617
U.S. Pat. No. 7,112,442
WO2010/082008

1. An apparatus for injection of a substance into a subject, comprising an assembly including a plurality of elongate non-hollow micro-nanoneedles forming a micro-nanoneedle patch for delivering the substance into the subject, at least some of said micro-nanoneedles have a non-uniform diameter with a wider upper end and a narrower lower end and a length from 5-100 um, wherein the upper end has a diameter from 1-20 um and the lower end has a diameter from 50-400 nm, wherein said micro-nanoneedles are spaced apart with appropriate spacing therebetween for minimizing multiple micro-nanoneedles penetrating one cell, and wherein said micro-nanoneedles are made from a relatively rigid material allowing the micro-nanoneedles to maintain rigidity during penetration and are configured to allow coating of the substance on surface of the lower end.

2. An apparatus as claimed in claim 1, wherein the substance is a bio-agent or is selected from a group including drug molecules, gene molecules such as plasmid DNA, siRNA, miRNA, shRNA, nanoparticles and nanowires.

3. An apparatus as claimed in claim 1, wherein the subject is a sample of cells collected on a substrate.

4. An apparatus as claimed in claim 3, wherein the substrate is a multi-well array of container.

5. An apparatus as claimed in claim 1, wherein said micro-nanoneedles are configured to adopt a generally conical profile.

6. An apparatus as claimed in claim 1, wherein the lower end of said micro-nanoneedles is pointy and/or sharp.

7. An apparatus as claimed in claim 1, wherein the lower end has a diameter from 50-100 nm.

8. An apparatus as claimed in claim 1, wherein each said micro-nanoneedle is spaced apart from an adjacent micro-nanoneedle by a distance from 5-50 um.

9. An apparatus as claimed in claim 1, wherein the micro-nanoneedles are fabricated from diamond, silicon, or any other rigid materials.

10. An apparatus as claimed in claim 1, wherein the micro-nanoneedle patch has a micro-nanoneedle density of 10⁵-10⁶ micro-nanoneedles per cm².

11. An apparatus as claimed in claim 1, comprising means for applying said micro-nanoneedle patch to the subject at a speed of 0-5 m/s.
